(19) United States
(12) Patent Application Publication
(10) Pub. No.: US 2026/0053677 A1
(43) Pub. Date: Feb. 26, 2026

(54) FORMING STATION FOR FORMING AN ABSORBENT CORE FOR DISPOSABLE HYGIENE PRODUCTS

(71) Applicant: TEKNOWEB CONVERTING SRL, PALAZZO PIGNANO (CR) (IT)

(72) Inventor: Pierangelo MANDOTTI, PALAZZO PIGNANO (CR) (IT)

(21) Appl. No.: 19/103,510

(22) PCT Filed: Jul. 19, 2023
(86) PCT No.: PCT/IT2023/050167

§ 371 Date: Feb. 12, 2025

(30) Foreign Application Priority Data
Aug. 8, 2022 (IT) 102022000016908

(51) Int. Cl. A61F 13/15

(52) U.S. Cl. CPC A61F 13/15642; A61F 13/15764; A61F 2013/15926

(57) ABSTRACT

The invention concerns the sector of production and packaging lines for disposable hygiene products, for example nappies and/or absorbent underpads for babies, adults and animals. In further detail, the invention concerns a forming station (1) for forming an absorbent core for disposable hygiene products of the type comprising at least one ply (2) scattered with cellulose. Said forming station (1) comprises:—a frame (3);—means for feeding cellulose flakes adapted to convey a flow of cellulose flakes towards said ply, and means for feeding a ply (2) in a feed direction (x);—a perforated belt (5) adapted to support said ply as it is fed forward;—a suction hood (6), positioned below said belt (5), adapted to suck said cellulose, causing it to lie flat on said ply (2);—a plurality of horizontal axis rotating brushes (7), arranged transversally to the feed direction (x) of said ply (2);—a screen (8) comprising a plurality of holes (8′) arranged between said brushes (7) and said ply (2);—“come and go” alternating movement means for said screen (8) parallel to the feed direction (x) of said ply (2); wherein said brushes (7) are functionally in contact with said screen (8) and said cellulose flakes are adapted to pass through said holes (8′) of said screen (8) facilitated by said suction hood (6) and by the combined movements of said brushes (7) and said screen (8).

### TECHNICAL FIELD OF APPLICATION

The invention concerns the sector of production and packaging lines for disposable hygiene products, for example nappies and/or absorbent underpads for babies, adults and animals.

In further detail, the invention concerns a forming station for forming an absorbent core for disposable hygiene products of the type comprising at least one ply scattered with cellulose.

### PRIOR ART

The forming stations for forming absorbent cores receive the cellulose flakes produced by a grinding mill and, with the help of a suction hood, distribute them on a ply, also made of cellulose, which is unwound and fed in a feed direction.

A forming station of known type comprises:
a frame;
means for feeding cellulose flakes comprising at least one duct adapted to convey a flow of cellulose flakes towards said ply;
a perforated belt adapted to support said ply as it is fed forward;
a suction hood, positioned below said belt, adapted to suck said cellulose causing it to lie flat on said ply;
a rotating levelling brush, positioned downstream of said duct through which the cellulose is fed and above said ply so as to lightly touch it, adapted to level the layer of cellulose flakes just deposited on said ply.

Based on different possible applications, said suction hood can be of the rotary or flat belt type, consequently modifying the geometry and distribution of the various mechanical components, while maintaining the same functions.

Said forming stations have some limits and drawbacks, particularly evident in production lines that are becoming faster and faster, and for the formation of particularly thin absorbent cores.

A first drawback derives from the fact that the cellulose flakes taken from the grinding mill, due to the turbulent motion created by suction of the hood, tend to become electrostatically charged and clump together when deposited on the ply.

Consequently, the cellulose deposits on the ply in a non-uniform manner: empty spaces alternate with clumps of cellulose which it is difficult for the levelling brush located downstream to distribute.

In any case, a huge expenditure of energy is required of said levelling brush, with consequent increase in consumption.

Said drawbacks are more evident for thinner and therefore very light absorbent cores (weighing less than 15 g/m2).

### PRESENTATION OF THE INVENTION

The invention aims to overcome these limits, providing a forming station for forming an absorbent core for disposable hygiene products that uniformly distributes the cellulose flakes, also with minimum thicknesses and gram weights, and is efficient, accurate, and speeds up production.

A further object of the present invention is to obtain an absorbent core which:
is thin, to facilitate subsequent packaging of the hygiene product that contains it, to reduce the volume of the finished packages, optimise use of the warehouse space and reduce transport costs;
is provided with a uniform cellulose layer so as to be uniformly absorbent;
has high quality and finish, to reduce user discomfort.

Said objects are achieved by a forming station for forming an absorbent core for disposable hygiene products of the type comprising at least one ply scattered with cellulose, where said forming station comprises:
a frame;
means for feeding cellulose flakes comprising at least one duct adapted to convey a flow of cellulose flakes towards said ply;
means for feeding a ply in a feed direction;
a perforated belt adapted to support said ply as it is fed forward;
a suction hood, located below said belt, adapted to suck said cellulose causing it to lie flat on said ply,
characterised in that it comprises:
a plurality of horizontal axis rotating brushes, arranged transversally to the feed direction of said ply;
a screen comprising a plurality of holes arranged between said brushes and said ply;
“come and go” alternating movement means for said screen parallel to the feed direction of said ply;
where said brushes are functionally in contact with said screen and said cellulose flakes are adapted to pass through said holes of said screen facilitated by said suction hood and by the combined movements of said brushes and said screen.

According to a first aspect of the invention, said forming station comprises a collection unit of said cellulose, arranged between said screen and said perforated belt, and closed peripherally so that it can be placed under a vacuum by said suction hood.

In particular, said collection unit comprises a first pair of lateral walls parallel to the feed direction of said ply and a second pair of lateral walls orthogonal to said feed direction.

Advantageously, said forming station comprises adjustment means for adjusting the reciprocal position of the said lateral walls parallel to the feed direction of said ply, to vary the dimension of said collection unit according to the width of the ply.

According to further aspects of the invention, each lateral wall orthogonal to said feed direction comprises a roller adapted to rest on said ply to seal said collection unit, allowing said ply to be fed forward.

Advantageously, said collection unit comprises a sealing system with scraper blades positioned in contact with each roller and ensuring the airtightness of said collection unit.

According to a possible embodiment, said “come and go” alternating movement means of said screen comprise at least a connecting rod and crank mechanism.

In particular, said crank is made of a wheel comprising, along a radius thereof, selective fixing means for said connecting rod, in order to adjust the range of the “come and go” alternating movement of said screen.

Advantageously, said frame comprises tracks and said screen comprises wheels adapted to cooperate with said tracks.

In a preferred embodiment, said suction hood comprises an extractor, a horizontal conveyor and a vertical conveyor joined to one another by means of a flange.

According to a further aspect, said horizontal conveyor comprises a suction inlet on which said perforated belt runs, said ply resting on the latter, and said suction inlet comprises blade shuttering means for adjusting the opening thereof.

In a possible variation of the invention, said horizontal conveyor and vertical conveyor each comprise a plurality of inner walls that divide them into horizontal chambers and vertical chambers respectively.

Even more preferably said horizontal conveyor and said vertical conveyor comprise shutters adapted to regulate the suction flow of said chambers, and control means for said shutters that can be operated externally.

In a possible variation of the invention, said cellulose flake feeding means comprise two ducts, arranged in sequence in the feed direction of said ply; a feed duct for feeding super-absorbent material is interposed between said ducts.

In particular, said screen comprises a central opening directly connected to said feed duct for feeding super-absorbent material, thus favouring the direct deposition thereof between a first and a second layer of cellulose flakes.

The invention has numerous advantages.

The rotation movement of the brushes combined with the “come and go” alternating movement of the screen favours uniform distribution of the cellulose flakes on the ply, facilitated by the suction of the suction hood.

The rotating brushes lightly touch the surface of the screen, accompanying the cellulose flakes towards the holes of the screen.

The “come and go” alternating movement of the screen guarantees uniform distribution of the cellulose, thus obtaining a substantially constant layer thereof on the ply.

The uniform thickness of the cellulose results in a layer without lumps or empty spaces, with the advantage of obtaining a final core with uniform absorbent capacity.

Advantageously, with a correct distribution of the cellulose, the required absorbent power being equal, it is possible to obtain thinner and lighter cores, with a weight in the order of 10-15 g/m2.

A possible change of screen, with appropriately-sized holes, allows different quantities of cellulose to be dosed, without having to intervene on other components of the forming station.

The levelling brush traditionally positioned downstream of the cellulose feed duct is no longer required.

The forming station can be easily adapted to the format of the ply.

The suction system can be easily shuttered to optimise the vacuum flow.

### BRIEF DESCRIPTION OF THE DRAWINGS

The advantages of the invention will become clearer from the following description, which describes a preferred embodiment, by way of non-limiting example, and with the help of the figures in which:

FIGS. 1 and 2 show, in axonometric view and in longitudinal section along a vertical plane respectively, a forming station for forming an absorbent core for disposable hygiene products according to the invention;

FIG. 3 shows, in axonometric view from below, part of the forming station of FIG. 1;

FIGS. 4 and 5 show, in axonometric view from two different angles, details of the forming station according to the invention;

FIG. 6 shows, in section, a detail of FIG. 5;

FIG. 7 shows, in axonometric view, a component of the forming station of FIG. 1;

FIG. 8 shows a detail of the section of FIG. 2;

FIG. 9-13 show, in axonometric view, components of the forming station, according to a possible variation of the invention.

### DETAILED DISCLOSURE OF A PREFERRED EMBODIMENT OF THE INVENTION

With reference to the Figures, a forming station 1 for forming an absorbent core for disposable hygiene products such as, for example, nappies or absorbent underpads for children, adults or animals, is illustrated.

Said absorbent core is of the type comprising at least a ply 2 scattered with cellulose flakes. In possible variations, said absorbent core also comprises granules of super-absorbent material, retained between said cellulose.

Said ply 2, also made of cellulose, is normally supplied in reels and unwound upstream of the forming station 1.

Said cellulose flakes are produced by a grinding mill also located upstream of the forming station 1.

The forming station 1 illustrated is of the flat chamber type, but the same characteristics can apply also to drum forming stations, which represent a variation in which the perforated belt runs on a drum placed in a vacuum and the forming chamber is shaped to adapt to the drum.

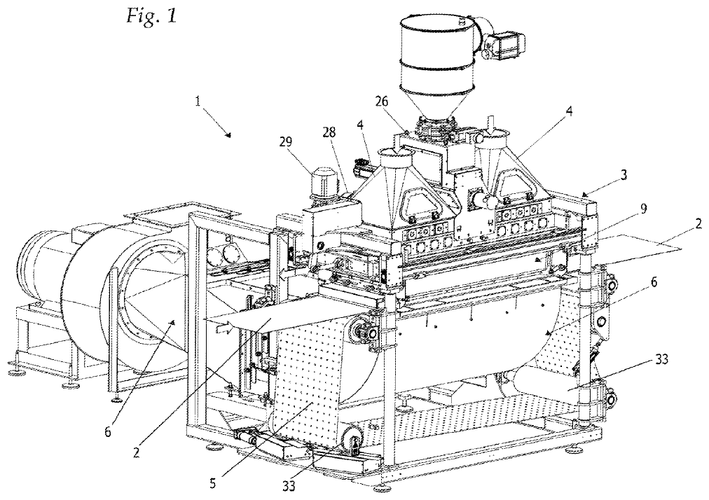

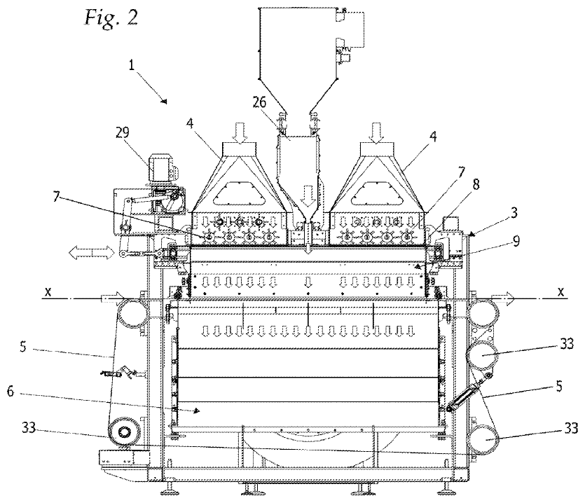

With reference to the assembly FIGS. 1-2, said forming station 1 comprises essentially:
a frame 3;
means for feeding cellulose flakes comprising at least one duct 4, or rather two ducts 4, adapted to convey a flow of cellulose flakes towards said ply 2;
a perforated belt 5 adapted to support said ply 2 as it is fed forward in a direction x;
a suction hood 6, positioned below said perforated belt 5, adapted to suck said cellulose, causing it to lie flat on said ply 2;
a plurality of horizontal axis rotating brushes 7, arranged transversally to the feed direction x of said ply 2;
first motor means 28 for said brushes 7;
a screen 8 comprising a plurality of holes 8′ arranged between said brushes 7 and said ply 2;
“come and go” alternating movement means for said screen 8 parallel to the feed direction x of said ply 2;
second motor means 29 for said screen 8;
a collection unit 9 of said cellulose, arranged between said screen 8 and said perforated belt 5, and peripherally closed so that it can be placed under a vacuum by said suction hood 6.

Said brushes 7 are functionally in contact with said screen 8 so as to lightly touch it with their ends, and each of them is provided with their own motor means 28.

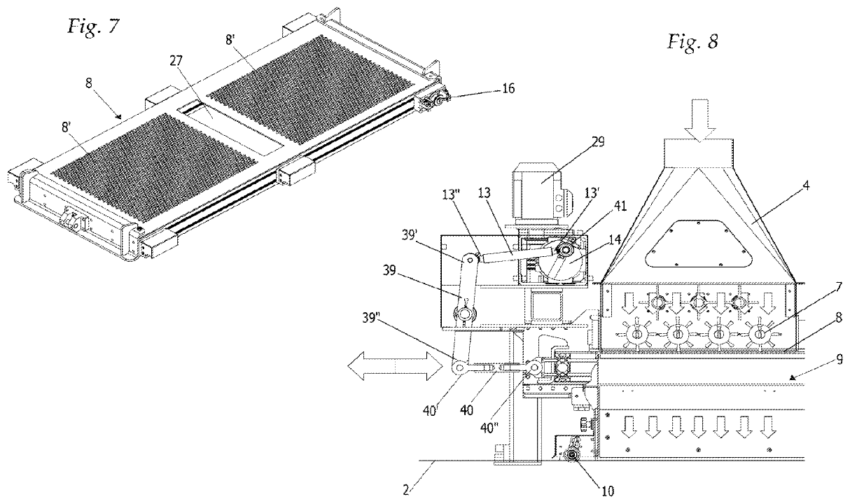

Substantially, said brushes 7 are adapted to lightly touch said screen 8 during their rotation movement, while the holes 8′ of said screen 8, illustrated in the detail of FIG. 7, allow passage of the cellulose from the feed ducts 4 to the collection unit 9, until reaching the ply 2.

Said cellulose flakes are adapted to pass through said holes 8′ of said screen 8 facilitated by said suction hood 6 and by the combination of the rotary movement of said brushes 7 and the “come and go” alternating movement of said screen 8.

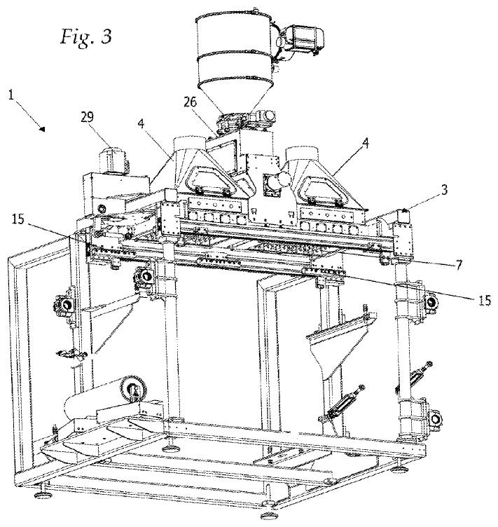

With particular reference to FIG. 3, said frame 3 is adapted to create a supporting and fixing structure, for example for the two ducts 4 in which the flow of cellulose flakes flows, for the rotating brushes 7, and for the motor means 28, 29.

Said ducts 4 are arranged in sequence in the feed direction x of said ply 2.

Between said ducts 4 for passage of the flow of cellulose flakes, a feed duct 26 is positioned for superabsorbent material which in some cases can be contained in the absorbent core itself.

Said ply 2 is fed in direction x supported by a perforated belt 5 closed in a ring and rotated by a plurality of motorized or relay rollers 33.

Said belt 5 is perforated to allow suction by the hood 6 below.

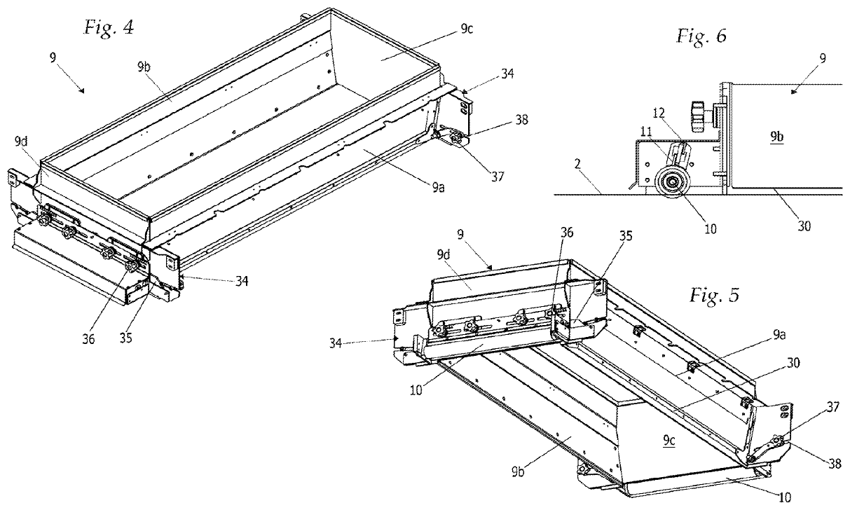

With particular reference to FIGS. 4-6, said collection unit 9 is illustrated, in which the uniformly distributed cellulose flakes are deposited on said ply 2.

Said collection unit 9 comprises a first pair of lateral walls 9a, 9b parallel to the feed direction x of said ply 2 and a second pair of lateral walls 9c, 9d orthogonal to said feed direction x, in addition to an assembly frame 34.

At least said lateral walls 9a, 9b parallel to the feed direction x comprise, along their edge facing said ply, a felt profile 30 which creates an airtight surround.

It must be possible for said suction hood 6 to place said collection unit 9 in a vacuum; the latter must therefore have an inner dimension corresponding to the actual dimension of the absorbent core to be produced and it must be airtight.

For said purposes, said forming station 1 comprises adjustment means for adjusting the reciprocal position of said lateral walls 9a, 9b parallel to the feed direction x of said ply 2, to vary the dimension of said collection unit 9 according to the width of the ply.

Said adjustment means, which act at both ends of said collection unit 9 in the feed direction x of said ply 2, comprise:
stop plates 35 movable and selectively fixable in a position by means of first handwheels 36, where said position predetermines the dimension, in particular the width, of said collection unit 9;
second handwheels 37 adapted to guide, along a cam profile 38 obtained on said assembly frame 34, the displacement of said lateral walls 9a, 9b parallel to the feed direction x of said ply 2 until touching said stop plates 35, and then fix the reciprocal position thereof.

For the purposes of the peripheral airtightness of said collection unit 9, each lateral wall 9c, 9d orthogonal to said feed direction x comprises an idle rubber-coated roller 10 adapted to rest, rolling, on said ply 2 (FIG. 5).

Said idle rubber-coated rollers 10 allow said collection unit 9 to be closed in an airtight manner, at the same time allowing feed of the ply 2 without the risk of tearing.

Said collection unit 9 also comprises a further seal system with scraper blades 11, 12; said blades 11, 12, the position of which is manually adjustable, behave like scrapers and are adapted to slide on said idle rubber-coated rollers 10 and create a seal against the sheet metal that forms the assembly frame 34 of said collection unit 9 (FIG. 6).

Said seal systems are essential to guarantee good suction of the cellulose flakes and prevent air gaps that could create turbulence such as to compromise uniform deposit of the cellulose on the ply 2.

With particular reference to FIG. 7, said screen 8 comprises two portions provided with holes 8′ for the passage of the cellulose flakes, arranged substantially below said cellulose feed ducts 4, and a central opening 27 directly connected to said feed duct 26 for feeding superabsorbent material, thus favouring direct deposition thereof on the ply 2, between a first and a second layer of cellulose.

Since said screen 8 moves with an alternating translation movement in the feed direction of the ply 2, said screen 8 comprises wheels 16 and said frame 3 comprises corresponding tracks 15 (FIG. 3) adapted to cooperate with said wheels 16.

With particular reference to the detail section of FIG. 8, said “come and go” alternating movement means of said screen 8 are illustrated.

In particular, said movement means comprise a mechanism with connecting rod 13 and crank 14.

Said connecting rod 13 comprises a first 13′ and a second 13″ end.

Said crank is produced by means of a wheel 14 on which a first end 13′ of said connecting rod 13 is fixed off-centre.

The second end 13″ of said connecting rod 13 is connected to said screen 8 by means of a linkage 39 of first type with two arms and a rod 40 with two hinges.

Said linkage 39 is hinged to said frame 3 and comprises a first 39′ and a second 39″ end.

Said first end 39′ of said linkage 39 is connected to the second end 13″ of said connecting rod 13.

Said second end 39″ of said linkage 39 is connected to a first end 40′ of said rod 40 with two hinges.

A second end 40″ of said rod 40 with two hinges is then connected to said screen 8.

To adjust the range of the “come and go” alternating movement of said screen 8, to modify the density of the cellulose deposited on the ply 2 and to take account of the final dimensions of the absorbent core, said crank 14 comprises, along a radius thereof, selective fixing means for the first end 13′ of said connecting rod 13.

Said selective fixing means comprise an adjustment screw 41 adapted to be moved along the operative radius of the crank 14: a shorter radius results in a reduced range of the alternating movement of the screen 8; vice versa, a longer radius results in a wider range of the alternating movement.

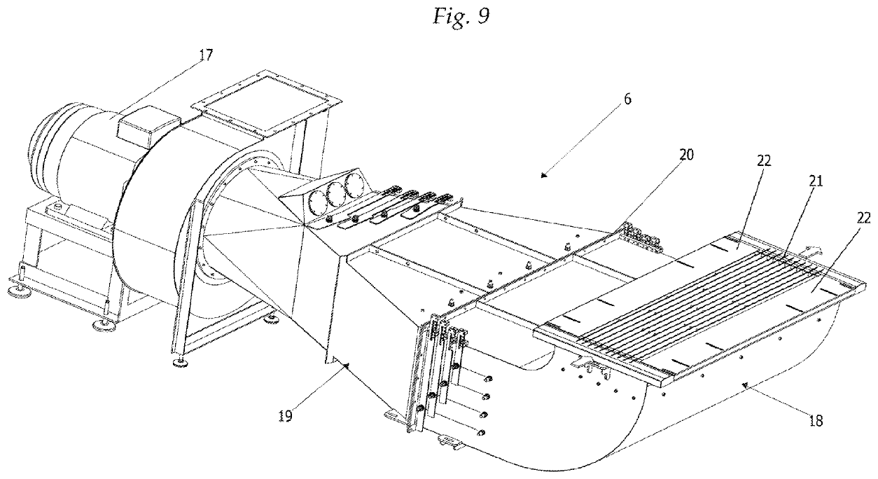

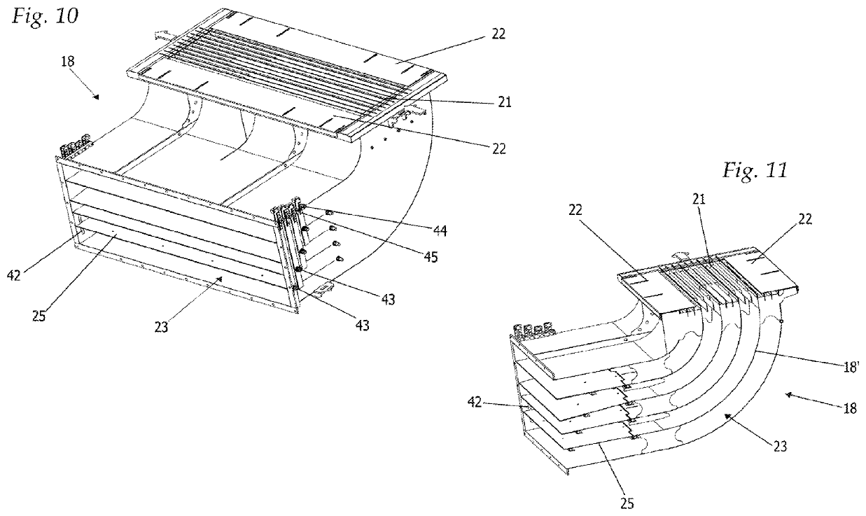

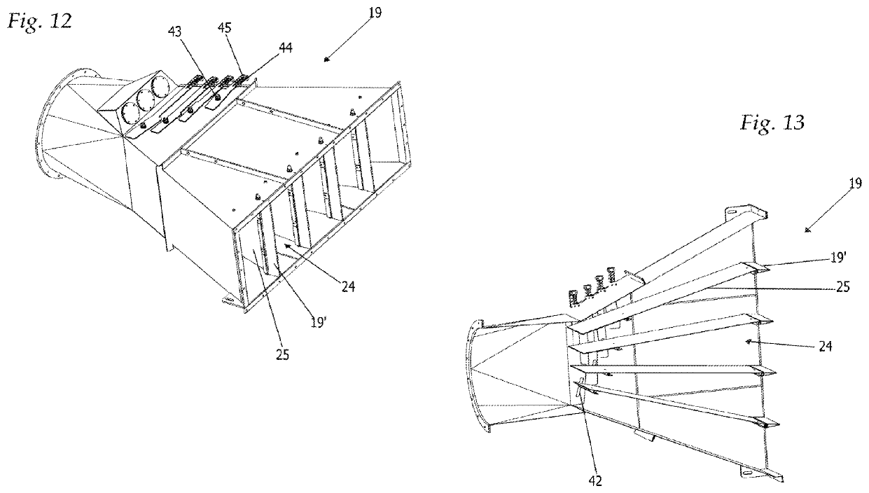

With particular reference to FIG. 9-13, said suction hood 6 is illustrated, positioned below the perforated belt 5 supporting the ply 2.

Said suction hood 6 comprises essentially an extractor 17 with horizontal axis, a horizontal conveyor 18 and a vertical conveyor 19, joined to one another by means of a flange 20.

Said horizontal conveyor 18 comprises a suction inlet 21 over which said perforated belt 5 runs, on which said ply 2 rests.

Said suction inlet 21 comprises blade shuttering means for adjusting the opening thereof.

Said blade shuttering means comprise two plates 22 sliding on the plane that contains said suction inlet 21 transversally to the feed direction x of said ply 2.

The sliding of said plates 22 narrows or widens said suction inlet 21 according to the width of the ply 2 being fed forward.

Said conveyors 18, 19 each comprise a plurality of inner walls 18′, 19′ that divide them into horizontal chambers 23 and vertical chambers 24.

Advantageously, said suction hood 6 can also vary its suction flow rate, selectively closing the section of said chambers 23, 24.

Said horizontal conveyor 18 and vertical conveyor 19 comprise shutters 25, revolvingly associated with said walls 18′, 19′ by means of hinges.

Said horizontal conveyor 18 and vertical conveyor 19 comprise control means for said shutters 25 that can be operated from the outside.

Said control means comprise, for each shutter 25:
a slot 42 obtained on an outer lateral wall of the respective conveyor 18, 19;
a control pin 43 associated with said shutter 25, passing through said slot 42 so that it protrudes on the outside of said suction hood 6, and sliding inside it;
locking means for locking a selected position of said control pin 43 in said slot 42.

For each control pin 43, said locking means comprise a pin 44 stably associated on the same outer lateral wall of the conveyor 18, 19 on which said slot 42 is obtained, and a comb 45, associated with said control pin 43, the teeth of which are adapted to cooperate with said pin 44 to maintain the position of the control pin 43 fixed inside the slot 42, and consequently to maintain fixed the inclination of the respective shutter 25 relative to the inner wall 18′, 19′ of the conveyor 18, 19 to which it is hinged.

The operation of the forming station 1 is as follows.

The ply 2 is fed into the forming station 1 in a feed direction x, forced by the suction hood to rest on said perforated belt 5.

The cellulose flakes, coming from a grinding mill, are blown into said feed ducts 4.

The cellulose flakes are sucked by said suction hood 6 facilitated by the combined rotation movement of the brushes 7.

As the brushes 7 rotate, they lightly touch said screen 8 and accompany the cellulose flakes through the screen holes 8′.

The simultaneous “come and go” alternating movement of the screen 8 ensures uniform distribution of the cellulose on the ply 2 below.

1. A forming station (1) for forming an absorbent core for disposable hygiene products of the type comprising at least one ply (2) scattered with cellulose, where said forming station (1) comprises:
a frame (3);
means for feeding cellulose flakes comprising at least a duct (4) designed to convey a flow of cellulose flakes towards said ply;
means for feeding a ply (2) in a feed direction (x);
a perforated belt (5) designed to support said ply (2) as it is fed forward;
a suction hood (6), positioned below said belt (5), designed to suck said cellulose, causing it to lie flat on said ply (2),
characterised in that it comprises:
a plurality of horizontal axis rotating brushes (7), arranged transversally to the feed direction (x) of said ply (2);
a screen (8) comprising a plurality of holes (8′) arranged between said brushes (7) and said ply (2);
“come and go” alternating movement means for said screen (8) parallel to the feed direction (x) of said ply (2);
wherein said brushes (7) are functionally in contact with said screen (8) and said cellulose flakes are designed to pass through said holes (8′) of said screen (8) facilitated by said suction hood (6) and by the combined movement of said brushes (7) and said screen (8).

2. The forming station (1) according to claim 1, characterised in that it comprises a collection unit (9) of said cellulose, arranged between said screen (8) and said perforated belt (5), and closed around the perimeter so that it can be placed under a vacuum by said suction hood (6).

3. The forming station (1) according to claim 2, characterised in that said collection unit (9) comprises a first pair of lateral walls (9a, 9b) parallel to the feed direction (x) of said ply (2) and a second pair of lateral walls (9c, 9d) orthogonal to said feed direction (x).

4. The forming station (1) according to claim 3, characterised in that it comprises adjustment means for adjusting the reciprocal position of said lateral walls (9a, 9b) parallel to the feed direction (x) of said ply (2), to vary the dimension of said collection unit (9) according to the width of the ply.

5. The forming station (1) according to claim 3, characterised in that each lateral wall (9c, 9d) orthogonal to said feed direction (x) comprises a roller (10) designed to rest on said ply (2) to seal said collection unit (9) allowing the ply to be fed forward.

6. The forming station (1) according to claim 5, characterised in that said collection unit (9) comprises a seal system with scraper blades (11, 12) positioned at each roller (10).

7. The forming station (1) according to claim 1, characterised in that said “come and go” alternating movement means of said screen (8) comprise at least a connecting rod (13) and crank (14) mechanism.

8. The forming station (1) according to claim 7, characterised in that said crank (14) is made of a wheel (14) comprising, along a radius thereof, selective fixing means for said connecting rod (13), in order to adjust the range of the “come and go” alternating movement of said screen (8).

9. The forming station (1) according to claim 1, characterised in that said frame (3) comprises tracks (15) and said screen (8) comprises wheels (16) designed to cooperate with said tracks (15).

10. The forming station (1) according to claim 1, characterised in that said suction hood (6) comprises an extractor (17), a horizontal conveyor (18) and a vertical conveyor (19) joined to one another by means of a flange (20).

11. The forming station (1) according to claim 10, characterised in that said horizontal conveyor (18) comprises a suction inlet (21) on which said perforated belt (5) runs, said ply (2) resting on the latter, and said suction inlet (21) comprises blade shuttering means (22) for adjusting the opening thereof.

12. The forming station (1) according to claim 10, characterised in that said horizontal conveyor (18) and vertical conveyor (19) each comprise a plurality of inner walls (18′, 19′) that divide them into horizontal chambers (23) and vertical chambers (24) respectively.

13. The forming station (1) according to claim 12, characterised in that said horizontal conveyor (18) and vertical conveyor (19) comprise shutters (25) designed to regulate the suction flow rate of said chambers (23, 24).

14. The forming station (1) according to claim 13, characterised in that said horizontal conveyor (18) and vertical conveyor (19) comprise control means for said shutters (25) that can be operated externally.

15. The forming station (1) according to claim 1, characterised in that said cellulose flake feeding means comprise two ducts (4), arranged in succession in the feed direction (x) of said ply (2); a feed duct (26) for feeding super-absorbent material is interposed between said ducts (4).

16. The forming station (1) according to claim 1, characterised in that said screen (8) comprises a central opening (27) directly connected to said feed duct (26) for feeding super-absorbent material, thus favouring the direct deposition thereof between a first and a second layer of cellulose flakes.
